(19) United States
(12) Patent Application Publication
(10) Pub. No.: US 2017/0231564 A1
(43) Pub. Date: Aug. 17, 2017

(54) PORTABLE PHYSIOLOGICAL PARAMETER DETECTION DEVICE AND PHYSIOLOGICAL PARAMETER DETECTION METHOD

(71) Applicant: BOE TECHNOLOGY GROUP CO., LTD., Beijing (CN)

(72) Inventors: Hao WANG, Beijing (CN); Defeng MAO, Beijing (CN)

(21) Appl. No.: 15/519,230

(22) PCT Filed: Apr. 7, 2016
(86) PCT No.: PCT/CN2016/078731

§ 371 Date: Apr. 14, 2017

(30) Foreign Application Priority Data
Jun. 10, 2015 (CN) 201510316547.7

(51) Int. Cl. A61B 5/00; A61B 5/024; A61B 5/021

(52) U.S. Cl. CPC A61B 5/6801; A61B 5/7225; A61B 5/0015; A61B 5/021; A61B 5/02438; A61B 2560/0214

(57) ABSTRACT

A portable physiological parameter detection device comprises: a detection module, configured to detect physiological parameters of a human body, and a power supply module, configured to supply power to the detection module, wherein the power supply module comprises a power generation unit, the power generation unit comprises two electrode layers arranged opposite to each other, an insulation layer is provided on each of surfaces that face each other of the two electrode layers, and the two insulation layers are in close contact with each other. The detection device produces relatively low pollution when being discarded. A portable physiological parameter detection method is further provided.

### TECHNICAL FIELD

Embodiments of the present disclosure relate to a portable physiological parameter detecting device and a physiological parameter detecting method.

### BACKGROUND

At present, portable physiological parameter detecting devices for detecting user's physiological parameters (heart rate, blood pressure, blood oxygen content etc.) are getting popular increasingly. This kind of devices is often incorporated on clothing or worn objects to detect human bodies with their own sensors and obtain physiological parameter data. They conduct data interactions with terminals as required and the terminals process and analyze data to realize real time monitoring of users' health condition.

However, in a prior art portable physiological parameter detecting device, an energy storing device such as a battery is generally set to provide electrical energy required for normal operation of the portable physiological parameter detecting device. However, since the endurance power of the above-mentioned energy storing device is insufficient for powering the portable physiological parameter detecting device for a long time, the user has to frequently charge the energy storing device (e.g., charging the battery) or replacing the energy storing device, which causes inconvenience for the user.

### SUMMARY

According to at least one embodiment of this disclosure, a portable physiological parameter detecting device is provided, comprising: a detecting module configured to detect physiological parameters of a human body; a power supply module electrically connected with the detecting module and configured to supply power to the detecting module, wherein the power supply module comprises an electricity generation unit comprising two electrode layers disposed oppositely with an insulating layer disposed on each of opposite surfaces of the two electrode layers, and the two insulating layers contact each other closely.

Optionally, opposite surfaces of the two insulating layers have same shape and size and the two insulating layers contact each other correspondingly according to the opposite surfaces' shape.

Optionally, the insulating layer is a piezoelectric material layer.

Optionally, the insulating layer is at least one of a polyimide layer, a phenylamine formaldehyde layer, a polyformaldehyde layer, a ethyl cellulose layer, a polyamide layer, a melamino-formaldehyde layer, a polyethylene glycol succinic acid ester layer, a cellulose layer or a cellulose acetic acid ester layer.

Optionally, the two insulating layers are of different materials.

Optionally, the electrode layer is at least one of an indium tin oxide layer, a graphene layer or a silver nanowire layer.

Optionally, the power supply module further comprises a voltage regulation unit electrically connected with the electricity generation unit for regulating a voltage generated by the electricity generation unit.

Optionally, the detecting module comprises: a sensing unit configured to detect physiological parameters of the human body and generate the physiological parameter information expressed in analog signals.

Optionally, the detecting module further comprises: a converting unit connected with the sensing unit and configured to convert the physiological parameter information expressed in analog signals into physiological parameter information expressed in digital signals.

Optionally, the device further comprising a storage module electrically connected with the power supply module and configured to store the physiological parameter information of the human body.

Optionally, the device further comprising a transmission module connected with the storage module, wherein the portable physiological parameter detecting device is connected with a terminal via the transmission module configured to transmit the physiological parameter information stored in the storage module to the terminal.

According to another embodiment of this disclosure, a physiological parameter detecting method is provided, which utilizes the portable physiological parameter detecting device, the physiological parameter detecting method comprises: detecting physiological parameters of a human body and generating physiological parameter information; wherein an electricity generation unit of the power supply module generates electricity by friction to supply electrical energy for normal operation of the portable physiological parameter detecting device.

Optionally, the physiological parameter detecting method further comprising: storing the physiological parameter information.

Optionally, the portable physiological parameter detecting device is in signal connection with a terminal, the method further comprising: transmitting the physiological parameter information to the terminal.

Optionally, the step of transmitting the physiological parameter information to the terminal comprises: determining whether there is any physiological parameter information that has not been transmitted to the terminal completely in a last transmission in addition to the physiological parameter information obtained in this time of detection; when there stored physiological parameter information that has not been transmitted to the terminal completely in the last transmission, transmitting the physiological parameter information that has not been transmitted to the terminal completely in the last transmission to the terminal, and then transmitting the physiological parameter information obtained in this time of detection to the terminal.

### BRIEF DESCRIPTION OF DRAWINGS

FIG. 1 is a structure block diagram of the portable physiological parameter detecting device provided in an embodiment of the present disclosure;

FIG. 2 is a structure diagram of an electricity generation unit provided in an embodiment of the present disclosure;

FIG. 3 is a structure diagram of a power supplying module shown in FIG. 1;

FIG. 4 is a structure diagram of a detecting module shown in FIG. 1;

FIG. 5a is another structure block diagram of the portable physiological parameter detecting device provided in an embodiment of the present disclosure;

FIG. 5b is a structure block diagram of the portable physiological parameter detecting device provided in an embodiment of the present disclosure that is connected with a terminal;

FIG. 6 is a flow chart of a physiological parameter detecting method provided in an embodiment of the present disclosure;

FIG. 7 is a flow chart of a physiological parameter information uploading method of the physiological parameter detecting method provided in an embodiment of the present disclosure;

FIG. 8 is a flow chart of a specific method of step 400 in FIG. 6; and

FIG. 9 is another flow chart of a physiological parameter detecting method provided in an embodiment of the present disclosure.

Reference numerals:

| | |
|---|---|
| 10-Power supply module | 11-electricity generation unit |
| 11a-Electrode layer | 11b-Insulating layer |
| 12-Voltage regulation unit | 20-Detecting module |
| 21-Sensing unit | 22-Converting unit |
| 30-Storage module | 40-Transport module |
| 800-Terminal | |

### DETAILED DESCRIPTION

In order to further explain the portable physiological parameter detecting device and the physiological parameter detecting method provided in the present disclosure, detailed description will be given below in connection with the accompanying drawings.

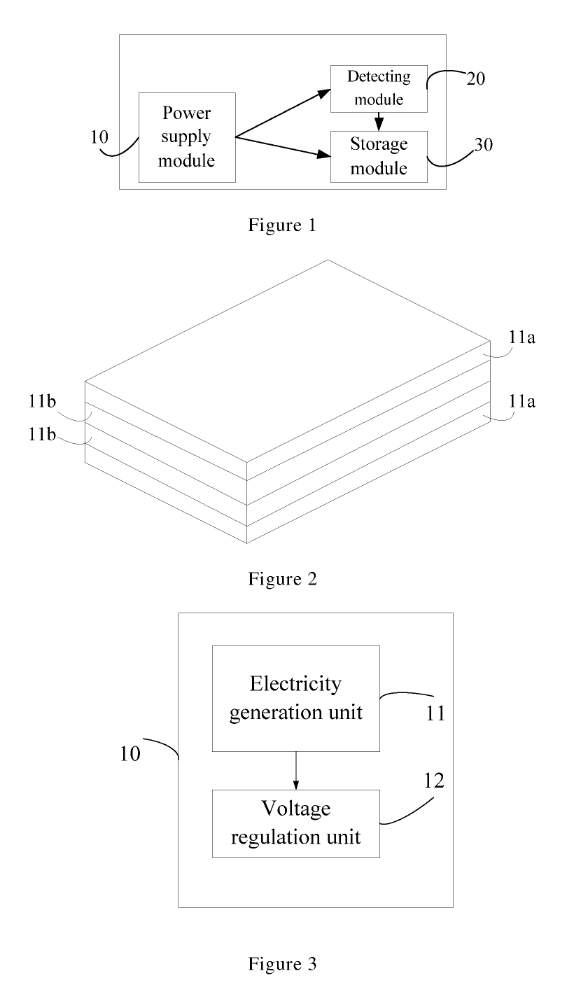

As shown in FIG. 1 and FIG. 2, the portable physiological parameter detecting device provided in an embodiment of the present disclosure includes: a detecting module 20 for detecting physiological parameter information of a human body; and a power supply module 10 electrically connected with the detecting module for powering the detecting module 20. Optionally, the portable physiological parameter detecting device may further include a storage module 30 with signal connection with the detecting module 20 for storing physiological parameter information of a human body to facilitate the user to view and analyze the detection results. The storage module 30 and the detecting module 20 are electrically connected with the power supply module 10 respectively. The power supply module 10 may include an electricity generation unit 11 including two electrode layers 11a disposed oppositely with an insulating layer 11b disposed on each of the opposite surfaces of the two electrode layers 11a respectively in which two insulating layers 11b contacting each other closely.

In an embodiment of the present disclosure, the electricity generation unit 11 may be for example a capacitor. The two electrode layers 11a serve as two electrodes of the capacitor respectively and the two insulating layers 11b serve as the insulating dielectric between the two electrodes in the capacitor.

In accordance with one example of the present disclosure, when the user presses the electricity generation unit 11, the two insulating layers 11b scrub each other on the interface where both of them contact each other closely such that the two insulating layers 11b carry static charges of opposite electrical properties respectively that move from the insulating layer 11b where they reside to the electrode layer 11a that contacts the insulating layer 11b (namely the capacitor is charged), thereby an electric potential difference occurring between the two electrode layers 11a and the capacitor being charged. When the user stops pressing the electricity generation unit 11, the laminated structure restores from the pressed state to the normal state and the two insulating layers 11b scrub each other to generate static charges with opposite electrical properties that move from the insulating layer 11b where they reside to the electrode layer 11a the insulating layer 11b contacts respectively such that an electric potential difference occurs again between the two electrode layers 11a.

In accordance with another example of the present disclosure, the insulating layer may also be made from a piezoelectric material. When the user presses the electricity generation unit 11, the piezoelectric material of the two insulating layers 11b is pressed to generate electric charges such that an electric potential difference occurs between the two electrode layers 11a, thereby charging the capacitor.

As can be known from the above, in the portable physiological parameter detecting device provided in embodiments of the present disclosure, the user presses the electricity generation unit 11 repeatedly to generate currents in the external circuit connected with the electricity generation unit 11, which in turn charges the portable physiological parameter detecting device continuously, thereby powering the detecting module 20 and the storage module 30 inside the device to work. As compared to energy storage devices with limited energy storage capability such as batteries, in embodiments of the present disclosure, the user can generate electrical energy continuously by pressing the electricity generation unit 11 repeatedly without recharging or replacing the energy storage device frequently, thereby improving the use convenience of the portable physiological parameter detecting device. As can be understood by those skilled in the art, in addition to pressing the electricity generation unit 11, pushing or scrubbing the electricity generation unit 11 with appropriate force can also make the electricity generation unit 11 generate electrical energy.

Furthermore, since the quantity of electricity generated by the two insulating layers in the electricity generation unit scrubbing each other is relative to the opposite contact area between the two insulating layers, as a preferred solution of embodiments of the present disclosure, the shapes and sizes of the opposite surfaces of the two insulating layers are identical and the two insulating layers contact each other entirely correspondingly according to the shapes of their opposite surfaces. With the above-mentioned settings, the area by which the two insulating layers contact each is made maximum, thereby increasing the quantity of electricity generated while they scrubbing each other, and in turn improving the endurance power of the portable physiological parameter device, and ultimately increasing the use convenience of the portable physiological parameter device.

In addition, in an embodiment of the present disclosure, since the electricity generation unit 11 is not a one-time energy storage device, it can generate electricity for many times with the user's activity to provide electrical energy to the detecting module 20 and the storage module 30. Therefore, the user does not need to replace the electricity generation unit 11 frequently, thereby not much wastes will be generated due to frequent abandonment of the electricity generation unit 11. And the electricity generation unit 11 generates electrical energy by physical electricity generation mode of triboelectricity, in which neither the electrode layer 11a nor the insulating layer 11b contains contaminants such as heavy metals, thereby resulting in less contamination when discarding the portable physiological parameter detecting device. Embodiments of the present disclosure are more energy-saving and environment-friendly than energy storage devices such as chemical cells, since while using one-time energy storage devices, the user needs to replace the one-time energy storage device frequently, resulting in more wastes. And common energy storage devices such as chemical cells contain multiple contaminants such as heavy metals which will cause much contamination while being discarded. Based on the above-mentioned reasons, the portable physiological parameter detecting device provided in embodiments of the present disclosure will not generate much wastes due to frequent abandonment of the electricity generation unit 11 and the contamination generated upon abandonment of the portable physiological parameter detecting device is relatively less.

Depending on the use of the user, the electricity generation unit 11 adopted in the embodiments of the present disclosure will have two modes for generating electrical energy. One of them is that the user actively presses the electricity generation unit 11 repeatedly to make the electricity generation unit 11 generate electrical energy. Another is to generate press or a certain degree of friction between the electricity generation unit 11 and other objects with the user's day to day activities, which in turn makes the electricity generation unit 11 generate electrical energy.

For the first electricity generation mode, it is possible to dispose the electricity generation unit 11 in the portable physiological parameter detecting device at a location that facilitates user's press and it is further preferred to identify the location. For example, when the portable physiological parameter detecting device provided in an embodiment of the present disclosure is integrated on a ring, it is possible to expose one electrode layer 11a of the electricity generation unit 11 outside of the ring and form special patterns such as a trademark on the exposed electrode layer 11a. Alternatively, a special color may be painted on the exposed electrode layer 11a such that the user can find the electricity generation unit 11 simply and quickly and press it to allow the electricity generation unit 11 to generate electrical energy more efficiently.

While for the second electricity generation mode, it is preferred to dispose the electricity generation unit 11 in the portable physiological parameter detecting device at a location where press or friction against another object is easy to occur. For example, when the portable physiological parameter detecting device provided in an embodiment of the present disclosure is integrated on clothings, it is possible to dispose the electricity generation unit 11 at a location where press or friction is easy to occur, such as the oxter, neckline or wristband of the clothings (the number of the electricity generation units is not limited) such that the electricity generation unit 11 can generate electrical energy as much as possible by making maximum utilization of the user's daily activity.

Since the portable physiological parameter detecting device is portable and its application environment is generally near the human body, based on the concept of easy-to-use and energy conserving, the embodiment of the present disclosure adopts the above-mentioned electricity generation mode that utilizes user's daily activities to make the electricity generation unit 11 generate electrical energy. On the other hand, there is no precise rules for the user's activities, which causes that it's impossible to ensure that the electricity generation unit 11 can generate electrical energy by utilizing user's activities that may be maintained until the physiological parameter information is saved completely. Therefore, the storage module 30 in the embodiment of the present disclosure may be but not limited to a memory device that would not lose data upon accident power breakdown such as a flash memory or a phase change memory. One skilled in the art can adopt proper memory devices as the storage module 30 according to practical situations.

For the above-mentioned implementations, the insulating layer 11b is generally made of a high molecular material, specifically a polyimide layer, a phenylamine formaldehyde layer, a polyformaldehyde layer, a ethyl cellulose layer, a polyamide layer, a melamino-formaldehyde layer, a polyethylene glycol succinic acid ester layer, a cellulose layer or a fibrin acetic acid ester layer etc. It is understood that the insulating layer 11b is not limited to the above-mentioned range of choice. Under the precondition that sufficient power generation is guaranteed, one skilled in the art can select other appropriate materials to make the insulating layer according to practical situations.

Furthermore, since the quantity of electric charges generated by friction between same material is less than that generated by friction between different materials, according to an example of the present disclosure, the two insulating layers 11b in the electricity generation unit 11 have different materials, which allows the electricity generation unit 11 to output large amount of electricity.

For the above-mentioned implementations, the electrode layer 11a is generally made of a material that has good electrical conductivity and steady physical properties and chemical properties. For example, the electrode layer 11a may be an indium tin oxide layer, a graphene layer or a silver nanowire layer. It is to be understood that the electrode layer 11a is not limited to the above-mentioned range of choice, one skilled in the art can select other proper materials to make the electrode layer 11a according to practical situations.

As shown in FIG. 3, In accordance with one example of the present disclosure, the power supply module 10 further includes a voltage regulation unit 12 electrically connected with the electricity generation unit 11 for regulating the voltage generated by the electricity generation unit 11. In embodiments of the present disclosure, the voltage regulation unit 12 may adopt micro-elements that regulating the voltage such as a voltage regulation capacitor, a voltage regulating diode, and a micro-voltage regulator. Regulating the voltage output from the electricity generation unit 11 by the voltage regulation unit 12 to make the power supply module 10 output steady voltage enables the detecting module 20 and the storage module 30 to operate under steady voltage and in turn prolong the service life of the portable physiological parameter detecting device.

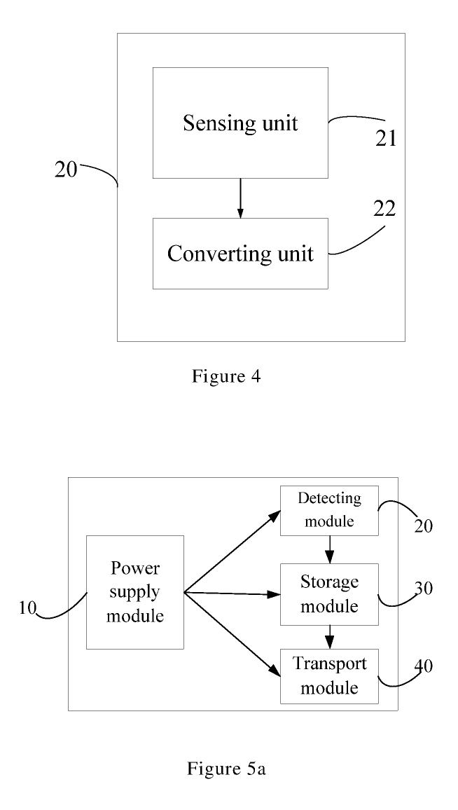

As shown in FIG. 4, in accordance with another example of the present disclosure, the detecting module 20 includes a sensing unit 21 and a converting unit 22. The sensing unit 21 is configured to detect physiological parameters of a human body and generate physiological parameter information expressed by analog signals. The converting unit 22 is in signal connection with the sensing unit 21 and is configured to convert the physiological parameter information expressed by analog signals into physiological parameter information expressed by digital signals. The sensing unit 21 provided in the present disclosure may include common sensors for measuring physiological parameters such as a blood pressure sensor and a heartbeat sensor. Since this kind of sensors generally output analog signals, while the time taken for the analog signals to be stored and transmitted is greater than the time taken for the corresponding digital signals to be stored and transmitted, in embodiments of the present disclosure, the converting unit 22 converts the physiological parameter information expressed in analog signals output by the sensing unit 21 into digital signal expression to facilitate storage and transmission of physiological parameter information. It is to be noted that since analog signals have high fidelity, physiological parameter information expressed in analog signals is still kept for some application scenarios that need precise physiological parameters such as population prone to high risk diseases. Based on the above-mentioned reasons, one skilled in the art can set a suitable signal type of the physiological parameter information according to practical situations.

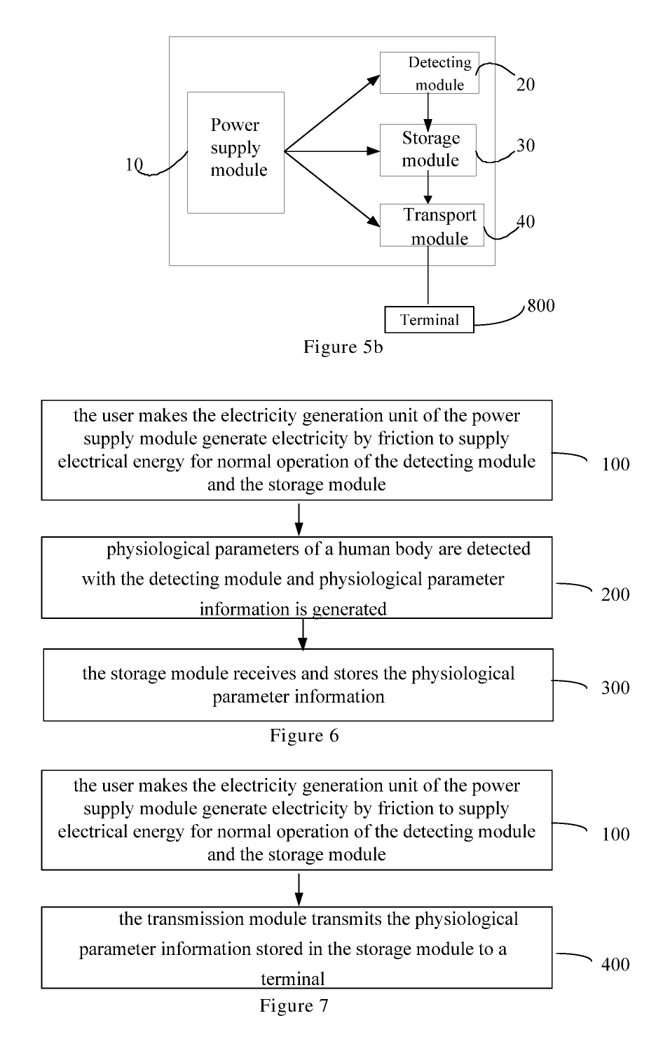

As shown in FIGS. 5a and 5b, in accordance with one example of the present disclosure, the portable physiological parameter detecting device may be connected with at least one terminal via a communication link. The terminal may be for example an electronic equipment such as a mobile terminal, a notebook computer or a tablet computer. The portable physiological parameter detecting device further includes a transmission module 40 in signal connection with the storage module 30 for transmit the physiological parameter information stored in the storage module 30 to the above-mentioned terminal. In the present embodiment, a communication connection between the portable physiological parameter detecting device and the terminal is established via the transmission module 40 to upload the physiological parameter information to the terminal. The above-mentioned communication connection may be a wired connection or a wireless connection (including a wireless compatibility authentication connection wifi, a Bluetooth connection, a infrared connection or a ultrasound signal connection etc.). Furthermore, a special application software may be configured on the terminal corresponding to the portable physiological parameter detecting device with which the terminal can acquire physiological parameter information from the portable physiological parameter detecting device more conveniently and further analyze, compare and predict the human health state.

It is to be understood that in embodiments of the present disclosure, it is possible to implement special designs on the exterior of the portable physiological parameter detecting device to inform the user the current job progress condition of the physiological parameter detection. For example, when the physiological parameter detecting device provided in the embodiment of the present disclosure is integrated on a hand ring, a progress bar window may be provided on the exterior of the ring which has different progresses corresponding to different stages such as detection, storage or transmission of the physiological parameters, and can also display the current quantity of electricity, thereby enabling the user to decide whether or not to continue making the electricity generation unit 11 generate electrical energy to maintain normal operation of the detecting module 20, the storage module 30 and the transmission module 40.

In an embodiment of the present disclosure, the electrical energy required for normal operation of the detecting module and the storage module is provided by the electricity generation unit, while the electrical energy provided by the electricity generation unit comes from the user's press or push on the electricity generation unit. As compared with the limited energy storage of energy storage device such as battery, in the present disclosure, the user may allow the electricity generation unit to generate electrical energy continuously as required. Therefore, it is not required to recharge or replace the energy storage device frequently, thereby improving the use convenience of the portable physiological parameter detecting device. Furthermore, since the electricity generation unit of the present disclosure provides electrical energy required for normal operation to the detecting module and the storage module by the physical electricity generation mode of triboelectricity, as compared to the chemical electricity generation mode of energy storage device such as battery in prior art portable physiological parameter detecting device, the electricity generation unit of the present disclosure does not contain contaminants such as heavy metals. Therefore, the portable physiological parameter detecting device provided in the present disclosure results in less contamination upon abandonment.

As shown in FIG. 6, the present disclosure further provides a physiological parameter detecting method using the portable physiological parameter detecting device described in any of the above-mentioned technical proposals which includes a detecting module, a storage module and a power supply module. The physiological parameter detecting method includes the following steps.

In step 100, the user makes the electricity generation unit of the power supply module generate electricity by friction to supply electrical energy for normal operation of the detecting module and the storage module. Specifically, the user presses or pushes the electricity generation unit to make the two insulating layers in the electricity generation unit scrub each other to generate static charges with opposite electrical properties respectively. Static charges with opposite electrical properties generated in individual insulating layer move to the electrode layer that contacts the insulating layer, such that an electric potential difference is generated between the two electrode layers, which in turn causes electric current in the external circuit connected to the two electrode layers. Electrical energy for normal operation is provided for the detecting module and the storage module by the generated current. In the above-mentioned electricity generation process, the user's press and push may be an act performed actively by the user (for example, the user's finger presses or pushes the location of electricity generation unit), or may be an unconsciously generated act of the user in daily activity (for example, press, friction generated between the electricity generation unit and the body, clothings etc. while the user is running).

In step 200, physiological parameters of a human body are detected with the detecting module and physiological parameter information is generated. In this step, the physiological parameter detecting device detects the human body with the sensing unit (which may include a blood pressure sensor, a heartbeat sensor etc.) in the detecting module to obtain various physiological parameters. The above various physiological parameters and information such as the time when the physiological parameters are detected are integrated into physiological parameter information. In general, the physiological parameter information obtained by the sensing unit is analog signals while analog signals have the features of large amount of information and high fidelity and take long time for storage and transmission as compared to digital signals. Therefore, when the user has high requirements on the accuracy degree of physiological parameters, it is possible to allow the detecting module to directly output physiological parameter information expressed by analog signals. When the user has low requirement on the accuracy degree of physiological parameters, it is possible to provide a converting unit in the detecting module by which physiological parameter information expressed by analog signals is converted into physiological parameter information expressed by digital signals to reduce the time taken to store and transmit it and in turn reduce the electrical energy consumption in its storage and transmission process.

In step 300, the storage module receives and stores the physiological parameter information.

In the physiological parameter detecting method provided in the embodiment of the present disclosure, the electrical energy is provided to the detecting module and the storage module by the user's press or push on the electricity generation unit. As compared with the limited energy storage of power supply device such as battery, in embodiments of the present disclosure, the user may allow the electricity generation unit to generate electrical energy continuously as required. Therefore, it is not required to recharge or replace the energy storage device frequently, thereby improving the use convenience of the portable physiological parameter detecting device.

Furthermore, since the physiological parameter detecting method provided in the embodiment of the present disclosure adopts physical electricity generation mode of triboelectricity to provide electrical energy required for normal operation of the detecting module and the storage module, as compared to the chemical electricity generation mode in energy storage device such as battery, the electricity generation unit used in the physiological parameter detecting method provided in the embodiment of the present disclosure does not contain contaminants such as heavy metal. Therefore, the physiological parameter detecting method provided in the embodiment of the present disclosure causes less contamination while abandoning the physiological parameter detecting device.

Furthermore, in the physiological parameter detecting method provided in the embodiment of the present disclosure, the portable physiological parameter detecting device is started to detect physiological parameters only when the user allows the electricity generation unit in the portable physiological parameter detecting device to generate electricity by friction, while in other time the portable physiological parameter detecting device is in an inactive state such as standby or sleeping mode. Therefore, as compared to the physiological parameter detecting method that maintains operation for a long time without a break, the physiological parameter detecting method provided in the embodiment of the present disclosure can save large amount of electrical energy.

As shown in FIG. 7, in accordance with another example of the present disclosure, after the step 100, said method may further include the following steps.

In step 400, the transmission module transmits the physiological parameter information stored in the storage module to a terminal connected with the portable physiological parameter detecting device. For example, the transmission module establishes a communication connection between the physiological parameter detecting device and the terminal (including wireless connection such as wireless consistency authentication connection wifi, infrared connection or ultrasound signal connection or wired connection), and upload the physiological parameter information stored in the storage module to the terminal that further analyzes, compares and predicts the human health state according to the physiological parameter information.

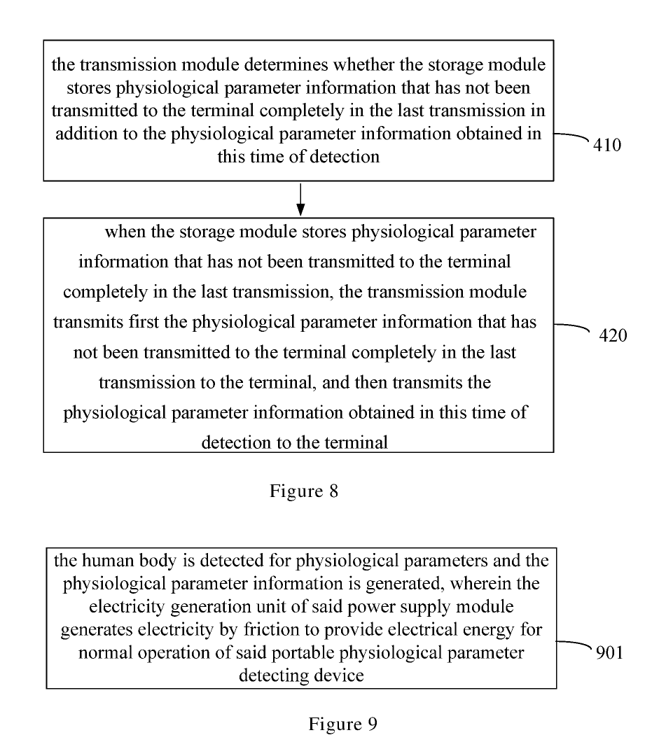

As shown in FIG. 8, in accordance with one example of the present disclosure, step 400 may includes the following steps.

In step 410, the transmission module determines whether the storage module stores physiological parameter information that has not been transmitted to the terminal completely in the last transmission in addition to the physiological parameter information obtained in this time of detection.

In step 420, when the storage module stores physiological parameter information that has not been transmitted to the terminal completely in the last transmission, the transmission module transmits first the physiological parameter information that has not been transmitted to the terminal completely in the last transmission to the terminal, and then transmits the physiological parameter information obtained in this time of detection to the terminal.

Since in the process that the transmission module uploads the physiological parameter information to the terminal, if the user stops pressing or pushing the electricity generation unit and causes accident power breakdown of the portable physiological parameter detecting device, the uploading of physiological parameter information will be incomplete or even error, in the physiological parameter detecting method provided in the embodiment of the present disclosure, with respect to steps 410 and 420, it is ensured that the physiological parameter information detecting device can completely upload the physiological parameter information obtained in every time detection to the terminal such that the terminal can better analyzes, compares and predicts the user's health state by determining, by the transmission module, whether the storage module stores any physiological parameter information that has not been transmitted to the terminal completely in the last transmission and while the determination result is yes, keeping transmitting the physiological parameter information that has not been transmitted to the terminal completely in the last transmission to the terminal.

Referring to FIG. 9, according to another example of the present disclosure, the physiological parameter detecting method may further include the following steps. In step S901, the human body is detected for physiological parameters and the physiological parameter information is generated, wherein the electricity generation unit of said power supply module generates electricity by friction to provide electrical energy for normal operation of said portable physiological parameter detecting device. That is, in case that the portable physiological parameter detecting device is out of power, the electricity generation unit of the power supply module may generate electricity to supply electrical energy for the portable physiological parameter detecting device. In addition, according to another example of the present disclosure, after detecting physiological parameters of the human body, it is also possible to store the physiological parameter information to facilitate viewing and analysis by the user.

In the description of the above-mentioned implementations, specific features, structures, materials or characteristics may be combined in proper manner in any one or more embodiments or examples.

The above are only specific implementations of the present disclosure. However the scope of the present disclosure is not limited thereto. Variations or substitutions that easily occur to any one skilled in the art within the technical scope disclosed in the present disclosure should be encompassed in the scope of the present disclosure. Therefore, the scope of the present disclosure should be based on the scope of the claims.

The present application claims priority of China Patent application No.201510316547.7 filed on Jun. 10, 2015, the content of which is incorporated in its entirety as part of the present application by reference herein.

1. A portable physiological parameter detecting device, comprising:
a detecting module configured to detect physiological parameters of a human body;
a power supply module electrically connected with the detecting module and configured to supply power to the detecting module, wherein the power supply module comprises an electricity generation unit comprising two electrode layers disposed oppositely with an insulating layer disposed on each of opposite surfaces of the two electrode layers, and the two insulating layers contact each other closely.

2. The portable physiological parameter detecting device of claim 1, wherein, opposite surfaces of the two insulating layers have same shape and size and the two insulating layers contact each other correspondingly according to the opposite surfaces' shape.

3. The portable physiological parameter detecting device of claim 1, wherein, the insulating layer is a piezoelectric material layer.

4. The portable physiological parameter detecting device of claim 1, wherein, the insulating layer is at least one of a polyimide layer, a phenylamine formaldehyde layer, a polyformaldehyde layer, a ethyl cellulose layer, a polyamide layer, a melamino-formaldehyde layer, a polyethylene glycol succinic acid ester layer, a cellulose layer or a cellulose acetic acid ester layer.

5. The portable physiological parameter detecting device of claim 1, wherein, the two insulating layers are of different materials.

6. The portable physiological parameter detecting device of claim 1, wherein, the electrode layer is at least one of an indium tin oxide layer, a graphene layer or a silver nanowire layer.

7. The portable physiological parameter detecting device of claim 1, wherein, the power supply module further comprises a voltage regulation unit electrically connected with the electricity generation unit for regulating a voltage generated by the electricity generation unit.

8. The portable physiological parameter detecting device of claim 1, wherein, the detecting module comprises:
a sensing unit configured to detect physiological parameters of the human body and generate the physiological parameter information expressed in analog signals.

9. The portable physiological parameter detecting device of claim 8, wherein, the detecting module further comprises:
a converting unit connected with the sensing unit and configured to convert the physiological parameter information expressed in analog signals into physiological parameter information expressed in digital signals.

10. The portable physiological parameter detecting device of claim 1, further comprising a storage module electrically connected with the power supply module and configured to store the physiological parameter information of the human body.

11. The portable physiological parameter detecting device of claim 10, further comprising a transmission module connected with the storage module,
wherein the portable physiological parameter detecting device is connected with a terminal via the transmission module configured to transmit the physiological parameter information stored in the storage module to the terminal.

12. A physiological parameter detecting method utilizing the portable physiological parameter detecting device of claim 1, the physiological parameter detecting method comprises:
detecting physiological parameters of a human body and generating physiological parameter information;
wherein an electricity generation unit of the power supply module generates electricity by friction to supply electrical energy for normal operation of the portable physiological parameter detecting device.

13. The detecting method of claim 12, the physiological parameter detecting method further comprising:
storing the physiological parameter information.

14. The physiological parameter detecting method of claim 12, wherein the portable physiological parameter detecting device is in signal connection with a terminal, the method further comprising:
transmitting the physiological parameter information to the terminal.

15. The physiological parameter detecting method of claim 14, wherein
the step of transmitting the physiological parameter information to the terminal comprises:
determining whether there is any physiological parameter information that has not been transmitted to the terminal completely in a last transmission in addition to the physiological parameter information obtained in this time of detection;
when there stored physiological parameter information that has not been transmitted to the terminal completely in the last transmission, transmitting the physiological parameter information that has not been transmitted to the terminal completely in the last transmission to the terminal, and then transmitting the physiological parameter information obtained in this time of detection to the terminal.
